(19) United States
(12) Patent Application Publication
(10) Pub. No.: US 2024/0343115 A1
(43) Pub. Date: Oct. 17, 2024

(54) System and Method for the Agile, Intuitive Control of Vehicle Functions

(71) Applicant: Bayerische Motoren Werke Aktiengesellschaft, Muenchen (DE)

(72) Inventor: Bernhard GASSER, Muenchen (DE)

(21) Appl. No.: 18/685,311

(22) PCT Filed: Aug. 18, 2022
(86) PCT No.: PCT/EP2022/073053

§ 371 Date: Feb. 21, 2024

(30) Foreign Application Priority Data
Sep. 22, 2021 (DE) 10 2021 124 586.9

(51) Int. Cl. B60K 35/10; A61B 5/024; B60K 35/81; B60K 35/85

(52) U.S. Cl. CPC B60K 35/10; A61B 5/024; B60K 35/81; B60K 35/85; B60K 2360/146; B60K 2360/148; B60K 2360/573; B60K 2360/5911

(57) ABSTRACT

A system and a method for agile, intuitive control of vehicle functions of a vehicle, include a wearable device which is configured to acquire sensor data. The sensor data comprise movement data and optical data relating to a user of the wearable device. The system has a computing unit which is configured to process the acquired sensor data; to determine a functional relationship between the processed sensor data and the vehicle; and to determine an assignment to a predefinable vehicle function taking the determined functional relationship into account. The vehicle has a control unit which is configured to control or adjust the predefinable vehicle function according to the determined assignment.

### BACKGROUND AND SUMMARY

The present invention relates to a system and a method for the agile, intuitive control of vehicle functions.

Added-convenience functions with respect to the operability of vehicle functions for the vehicle user are known. As such, for example what are known as added-convenience access systems are known that allow a vehicle to be unlocked and/or started without actively using the vehicle key, for which purpose the vehicle usually uses a radio-based system to locate the key relative to the vehicle. Another example of an added-convenience vehicle function is automatic or electric tailgate operation. This allows the vehicle tailgate to be opened and closed contactlessly by virtue of the vehicle having a built-in sensor system that detects a predetermined movement at the vehicle and, on detection, triggers the opening or closing of the tailgate. Additionally or alternatively, the vehicle and/or the vehicle key may have one or more installed operator control elements, operation of which results in the tailgate opening or closing. Furthermore, it is known practice to use appropriate operator control elements in the vehicle to allow further vehicle functions, such as e.g. opening or closing of the sliding/lifting roof, activation or deactivation of lighting elements, etc. Gesture control in the vehicle is also an example of an added-convenience vehicle function. For this purpose, for example an interior camera in the vehicle detects a predefined hand movement from a vehicle occupant. Depending on the detected hand movement, a vehicle function can be triggered that has a relative temporal relationship with one or more predefined vehicle functions such as e.g. changing station, taking or rejecting an incoming call, volume adjustment, etc. A disadvantage of this is that the aforementioned vehicle functions require expensive integration of the necessary sensors and/or operator control elements in the vehicle. Even retrofitting the aforementioned added-convenience functions is possible only with a high level of outlay, since the applicable sensors and/or operator control elements need to be retrofitted in the vehicle. A built-in sensor system has the additional disadvantage that additional hardware needs to be installed in the vehicle, for example a control unit with an appropriate housing and also an associated computing unit, leading to increased costs and, for example, environmental pollution during manufacture and disposal. Moreover, energy consumption becomes necessary in the vehicle.

The object of the invention is to provide a solution that allows improved, inexpensive, agile and intuitive provision of vehicle functions in the vehicle.

This object is achieved according to the invention by the features of the independent claims. Preferred embodiments are covered by the dependent claims.

The aforementioned object is achieved by way of a system for the agile, intuitive control of vehicle functions of a vehicle, comprising:
a wearable device designed to capture sensor data, the sensor data comprising motion data and optical data relating to a user of the wearable device;
a computing unit designed
   to process the captured sensor data;
   to determine a functional relationship of the processed sensor data with the vehicle; and
   to determine an association with a predefinable vehicle function in consideration of the determined functional relationship;
wherein the vehicle comprises a control unit designed to control or regulate the predefinable vehicle function in accordance with the determined association.

The system comprises at least one wearable device. Within the context of the document, the term wearable device, or wearable, or wearable computer system, covers in particular modern wearable computer systems that are worn on the body of the user of the wearable device during use, in particular smartwatches, but also smartglasses, smartbands, etc., which have a multiplicity of sensors capable of capturing motion data and optical data relating to the wearer of the wearable device and of using a communication unit to transmit them wirelessly—for example using an air or radio interface such as Bluetooth Low Energy (BLE) or the mobile radio network.

The wearable device comprises a sensor unit designed to capture sensor data. In particular, the sensor data may comprise motion data relating to the wearer of the wearable device. To capture the motion data, the sensor unit can capture sensor data from one or more of the following sensors:
an acceleration sensor, or accelerometer, which ascertains an acceleration by measuring an inertial force acting on a mass or test mass, with the result that it can determine the acceleration, a speed increase or decrease and/or a direction of movement of the wearable device; and/or
a position determination sensor, or position determination unit, for recording or determining the geographical position, or current position data, using a navigation satellite system. The navigation satellite system may be any established or future global navigation satellite system (GNSS) for position determination and navigation by receiving the signals from navigation satellites and/or pseudolites. By way of example, it may be the Global Positioning System (GPS), GLObal NAvigation Satellite System (GLONASS), Galileo positioning system and/or BeiDou Navigation Satellite System. In the example of GPS, the position determination sensor, or the position determination unit, may be a GPS module designed to determine current GPS position data relating to the wearable device; and/or
a gyro sensor, which is an acceleration or position sensor designed to record extremely small accelerations, rotational movements and/or a change of position of a mass or test mass. Data from the gyro sensor can be combined with position data from a navigation module, the combination of gyro sensor data and position data being able to be used to ascertain changes of direction, for example, very accurately; and/or
a magnetic field sensor designed to record a current alignment or direction of movement of the wearable device; and/or
a proximity sensor for activating and deactivating the display of the wearable device; and/or
a UWB sensor system that transmits a signal from the wearable device to multiple anchors, or UWB receivers, in the vehicle and is received on the vehicle at different times depending on the distance from the anchors, the computing unit being able to use this to determine the position of the wearable device relative to the vehicle; and/or
at least one further sensor designed to capture motion data from the wearable device.

The sensor data moreover comprise optical data relating to the wearer of the wearable device. To capture the optical data relating to the wearer of the wearable device, the sensor unit can capture sensor data from an optical sensor. The optical sensor emits light, for example in the green wavelength range, into the tissue, for example on the wrist, and measures the reflected light. The reflected light intensity varies with the pulsation of the blood vessels. This allows the heart rate of the wearer of the wearable device to be determined. In other words, the sensor unit may comprise an optical sensor for capturing heart rate data relating to a wearer of the wearable device. By way of example, the prior art discloses a function of smartwatches that allows a distinction to be made between two hand gestures by the hand wearing the smartwatch. The first hand gesture comprises bringing together the thumb and index finger; the second hand gesture comprises forming a fist.

The system comprises at least one vehicle. Within the context of the document, the term vehicle covers mobile means of transport used for transporting people (passenger transport), goods (freight transport) or tools (machines or implements). In particular, the term vehicle covers motor vehicles and also motor vehicles that may be at least partially electrically driven (electric car, hybrid vehicles).

The vehicle can be controlled by a vehicle driver. In addition or alternatively, the vehicle may be a vehicle driving in at least partially automated fashion. Within the context of the document, the term “vehicle driving in automated fashion” or “automated driving” can be understood to mean driving with automated longitudinal or lateral guidance or autonomous driving with automated longitudinal and lateral guidance. Automated driving may be for example driving for an extended period of time on the freeway or driving for a limited period of time when parking or maneuvering. The term “automated driving” covers automated driving with some level of automation. Illustrative levels of automation are assisted, partially automated, highly automated or fully automated driving. These levels of automation have been defined by the German Federal Highway Research Institute (BASt) (see BASt publication “Forschung kompakt”, issue 11/2012). In the case of assisted driving, the driver performs the longitudinal or lateral guidance on an ongoing basis, while the system undertakes the respective other function within certain boundaries. In the case of partially automated driving, the system undertakes the longitudinal and lateral guidance for a certain period of time and/or in specific situations, the driver needing to monitor the system on an ongoing basis as in the case of assisted driving. In the case of highly automated driving, the system undertakes the longitudinal and lateral guidance for a certain period of time without the driver needing to monitor the system on an ongoing basis; however, the driver must be capable of taking over vehicle guidance within a certain time. In the case of fully automated driving, the system may automatically cope with driving in all situations for a specific application; a driver is no longer needed for this application. The aforementioned four levels of automation correspond to SAE levels 1 to 4 of SAE standard J3016 (SAE-Society of Automotive Engineering). Furthermore, SAE J3016 also has provision for SAE level 5 as the highest level of automation, which is not included in the definition from the BASt. SAE level 5 corresponds to driverless driving, in which the system can automatically cope with all situations throughout the journey in the same way as a human driver.

The system comprises at least one computing unit. The computing unit is designed to process the captured sensor data. The processing of the sensor data can comprise a preprocessing of the raw sensor data.

In addition, the processing of the sensor data can comprise the determination of a predefinable, or predefined, movement of the user of the wearable device. Within the context of this document, these data are also called motion data. Examples of a predefined movement as motion data relating to the user of the wearable device may be a step to the side, a jump, a wave or any other predefinable, or predefined, movement that can be detected by the sensors.

The predefinable, or predefined, movement of the user of the wearable device can be determined using suitable machine learning algorithms, for example models created using machine learning methods—e.g. by way of supervised learning or unsupervised learning.

In addition or alternatively, the processing of the sensor data can comprise the determination of predefined, or predefinable, gestures of the user or wearer of the wearable device. The predefined gestures can be determined using the aforementioned motion data that can be captured by one or more of the aforementioned motion sensors, and also using the optical data from the optical sensor, or optical heart rate sensor, using suitable machine learning algorithms. In particular, the aforementioned sensors can be used to detect subtle differences in a muscle movement and in a tendon activity in the wearer of the wearable device. Such an approach is—as described hereinabove-known from the prior art. By way of example, similarly to Morse code or the Morse alphabet, in which letters are formed by stringing together short and long sound signals in some way, the two different hand gestures can represent any number of different vehicle functions by stringing together the aforementioned gestures “bringing together the thumb and index finger” and “forming a fist” in some way. With the described combinations with the movement gesture, or the functional relationship, the local relationship and the temporal relationship, the location on the vehicle and the temporal aspect, the possibilities are enormously diverse.

Advantageously, this allows the user of the wearable device to use subtle gestures—in the example of the smartwatch—by the hand wearing the smartwatch, for example pressing together the index finger and thumb, closing a fist, etc., to perform a predefined, or predefinable, movement with the least amount of effort.

A time limit for detecting the movement and/or gesture and/or some combination of a sequence of movements and/or gestures may also be imposed for determining the functional relationship. By way of example, the functional relationship may require the gesture/movement/random combination thereof to be completed within a predetermined period of time, otherwise a functional relationship is not detected. As such, by way of example, it may be predefined that a combination of gestures comprising “bringing together the thumb and index finger” in combination with “forming a fist” needs to be performed within 2 seconds in order to trigger the vehicle function “open trunk”.

The predefinable, or predefined, movement, or predefinable, or predefined, gesture thus determined—that is to say the determined functional relationship—may be associated with a predefinable, or predefined, vehicle function. In addition, some combination of a predefined movement, or predefined gesture, may be associated with a predefined vehicle function. By way of example, the user of the vehicle may stand behind the vehicle. The user of the vehicle may wear a smartwatch on one hand. The user of the vehicle may raise this hand to greet a neighbor. This hand movement would not be associated with a predefined vehicle function. If, however, the user of the vehicle first performs a predefined hand gesture with this hand and then raises this hand, for example, this combination of gesture and movement may be associated with the vehicle function “open trunk”.

In addition, the computing unit is designed to determine a functional relationship of the processed sensor data with the vehicle.

In particular, the functional relationship may arise as a result of one or more of the following conditions:
detected movement, or sequence of movements, of the user of the wearable device, as explained hereinabove;
detected gesture, or sequence of gestures, of the user of the wearable device, as explained hereinabove.

In addition, the computing unit is designed to determine—when a functional relationship has been detected—an association with a predefined vehicle function in consideration of the determined functional relationship. This can be done for example using a continually updatable association stored in a suitable storage unit.

The predefined, or predefinable, vehicle function may comprise one or more of the following vehicle functions: A. Outside, or exterior of, the vehicle:
open/close a charging flap, or a fuel filler flap, of the vehicle;
unlock a charging connector from a charging socket of the vehicle;
open/close a tailgate of the vehicle;
open/close some flap of the vehicle;
open/close one or more automatic doors of the vehicle;
control a boarding aid of the vehicle;
open/close a garage/an entrance gate using a radio interface of the vehicle;
open/close one or more windows of the vehicle;
open/close one or more sunblinds and/or shades of the vehicle;
open/close a sliding and lifting roof of the vehicle;
extend/retract a trailer hitch of the vehicle;
unlock/lock or secure the vehicle;
switch on/off auxiliary heating and/or auxiliary ventilation and/or auxiliary cooling of the vehicle;
switch on/off a parking light and/or sidelight and/or hazard light of the vehicle;
activate/deactivate some lighting effect of the vehicle;
trigger a camera recording (photograph or film);
activate/deactivate a sound from the vehicle;
actuate a horn of the vehicle;
adjust a seat position and/or steering column of the vehicle with respect to a personalized setting of the wearer of the wearable device;
unparking the vehicle;
command to stop the vehicle for garage parkers (automatic parking and unparking);
actuate a level adjustment of the vehicle;
trigger a parking/unparking process of the vehicle;
control a garage parker for automatically parking and etc.
B. Inside, or interior of, the vehicle:
scroll, swipe, select and/or slide in the vehicle interior, for example as a substitute for the gesture camera in the vehicle interior;
vehicle;
take or reject or terminate phone call;
change the volume of audio outputs in the vehicle;
activate/deactivate a display of a display unit in the
C. Head-up display:
activate/deactivate a function of the head-up display;
activate/deactivate a content that is output via the head-up display;
etc.
D. Status requests with respect to the vehicle:
request a state of charge of an energy store, for example a battery, of an at least partially electrically operated vehicle;
request a tire state of at least one tire of the vehicle;
request a general state of the vehicle;
request a status of the central locking of the vehicle;
request a status of a theft warning system of the vehicle;
request a tank fill level of a tank of the vehicle;
request a range with the current tank fill level and/or the current state of charge of the vehicle;
etc.

The aforementioned predefined, or predefinable, vehicle functions are listed merely by way of illustration; in principle, the approach described in this document allows any desired vehicle functions to be controlled or regulated and any desired statuses of the vehicle to be called up.

The vehicle comprises a control unit. The control unit is designed to control or regulate the predefinable vehicle function in accordance with the determined association.

Advantageously, it is therefore possible to perform particularly agile, flexible and intuitive control and/or regulation of a large number of vehicle functions without the need for expensive sensors and/or operator control elements in the vehicle. This facilitates better operator control of functions in and around the vehicle using the latest technologies in the field of wearable devices, which provide the necessary sensors anyway. Another important advantage is that the vehicle functions can be controlled or regulated hands-free, i.e. without input being required from the user of the wearable device using the device itself, since a gesture, for example a hand gesture, of a user of a smartwatch as the wearable device can trigger the aforementioned vehicle function(s) without the need for operator control of the smartwatch using the display. This is particularly convenient for the user of the wearable device. New vehicle functions can be added or adapted or modified as desired without the need for complex and expensive adaptations or installations in the vehicle.

Preferably, the vehicle and the wearable device each comprise a communication unit, the vehicle and the wearable device being designed to set up a Bluetooth Low Energy (BLE) connection to one another.

BLE is a radio technology that allows communication between two communication subscribers. BLE has very low power consumption compared with conventional Bluetooth.

By way of example, the wearable device may already be designed as a digital key, or digital vehicle key, for the vehicle in a manner known from the prior art. BLE technology therefore allows the wearer of the wearable device, without any initial input or request for an initial connection between the vehicle and the wearable device, to control or regulate the aforementioned vehicle function(s) hands-free by approaching the vehicle by using a gesture, security with respect to the communication between the vehicle and the wearable device being ensured at the same time by the digital key security requirements.

For the example of a smartwatch as the wearable device, it is therefore sufficient for the user of the smartwatch to perform a hand gesture in BLE communication range of the vehicle in order to trigger the aforementioned vehicle function(s). As such, by way of example, the user can form a first with the hand to which the smartwatch is attached in order to unlock and/or lock the vehicle. As another example, bringing together the thumb and index finger of the hand wearing the smartwatch can output a state of charge of an energy store of the vehicle on a display of the smartwatch.

Preferably, the computing unit is moreover designed to determine a local relationship of the processed sensor data with the vehicle, the determined local relationship being taken into consideration for determining the association with the predefinable vehicle function in consideration of the determined functional relationship. By way of example, the local relationship can be provided using ultra-wideband (UWB, see below) technology.

The data can be transferred from the wearable device to the vehicle using ultra-wideband (UWB) technology, for example. This is a short-range radio communication that uses extremely large frequency ranges with a bandwidth of at least 500 MHz or of at least 20% of the arithmetic mean of the lower and upper cutoff frequencies of the frequency band used. Advantageously, the use of UWB technology allows high-precision determination of the positions of the wearable device in relation to the vehicle to be achieved. The data can be transmitted from the wearable device to the vehicle locally using a suitable radio interface, e.g. Bluetooth Low Energy (BLE), or the mobile radio network. The local relationship can result from the high-precision determination of the position of the wearable device, or the wearer of the wearable device, in relation to the vehicle. The position of the wearable device in relation to the vehicle can be provided in zones in this case, depending on the system design. By way of example, a rear zone, a front zone and side zones are conceivable outside the vehicle. In addition, the local relationship can also be determined by way of a precise position of the wearable device with respect to the vehicle, for example 1 meter (m) in front of the driver's door or near to the charging flap. One alternative is for it to also be possible for the local relationship in the vehicle interior to be determined as a precise position in the vehicle interior. In another example, the vehicle interior can also be divided into zones, e.g. driver's seat, front-seat passenger's seat, backseat area on the right, etc. To determine the local relationship, the wearable device may comprise a UWB sensor system, or a UWB sensor, that transmits a signal from the wearable device to multiple anchors, or UWB receivers, in the vehicle, and is received on the vehicle at different times depending on the distance from the respective anchors, the computing unit using this to determine the position of the wearable device relative to the vehicle.

The inclusion of the local relationship increases the agility and flexibility of the control of the vehicle functions further because, in the example of the smartwatch as the wearable device, the wearer thereof can use a predefinable hand gesture, for example, to initiate opening of the driver's door while standing on the driver's side of the vehicle, to initiate opening of the front-seat passenger's door while standing on the front-seat passenger's side of the vehicle, to initiate opening of the trunk while standing at the rear of the vehicle, etc.

Preferably, the computing unit is moreover designed to determine a temporal relationship of the processed sensor data with the vehicle, the determined temporal relationship being taken into consideration for determining the association with the predefinable vehicle function in consideration of the determined functional relationship.

By way of example, the temporal relationship may comprise the determination of a duration of a gesture of the wearer of the wearable device and/or of a temporal variable, for example a start time and a stop time. In the example of the smartwatch, the wearer thereof can use a hand gesture, for example, to initiate opening of a vehicle window, relaxation of the hand gesture resulting in the process being terminated, which means that opening of the vehicle window is terminated when the hand gesture is relaxed. This increases the agility and flexibility of the control of the vehicle functions further.

In addition or alternatively, the temporal relationship can also be used to attain functional safety goals. A functional safety goal may require the correct functionality of a vehicle function to be assured along the whole process chain thereof. In a conventional vehicle electrical system communication, this may be an Alive signal. The Alive signal is a signal from the wearable device that cyclically changes its value in order to signal to the vehicle that the wearable device is still “alive”. This allows the vehicle to ensure that a signal status, for example relating to a hand gesture from the wearable device, is not “frozen” in the process chain.

For the example of the smartwatch, the wearer thereof can initiate the start of an action, for example an automated parking process for the vehicle, by forming a fist, for example. The functional safety goal may now require, during the automated parking process for the vehicle, as the functional safety goal, the wearer of the smartwatch to cyclically bring together their thumb and index finger and relax them again during the automated parking process in order to signal that the automated parking process is intended to be continued. If the aforementioned functional safety goal is not met, the automated parking process would be terminated for safety reasons. By forming a first again, the wearer of the smartwatch can trigger the end of the action, here the automated parking process. Such an approach demonstrates combinational control of vehicle functions.

Advantageously, the agility and flexibility of the control of the vehicle functions is therefore increased further, safety when controlling the vehicle functions being ensured at the same time.

Preferably, the local relationship moreover or alternatively comprises the detection of a direction of the motion data relating to the user of the wearable device, the detected direction being taken into consideration for determining the association with the predefinable vehicle function in consideration of the determined functional relationship.

For this purpose, the aforementioned UWB technology can be used. UWB technology allows high-precision determination of not only the positions of the wearable device in relation to the vehicle but also a direction of a movement of the wearable device in relation to the vehicle. By way of example, the direction of the motion data relating to the user of the wearable device can therefore be determined, or detected, by using UWB technology in a manner known from the prior art. In the example of the smartwatch as the wearable device, the wearer of the smartwatch can for example use a hand gesture and point to the rear door on the driver's side of the vehicle in order to initiate opening of the window on the rear driver's side door of the vehicle. By using the same hand gesture and pointing to the front driver's side door of the vehicle, the wearer can initiate opening of the window of the front door on the driver's side of the vehicle.

Advantageously, the agility and flexibility for controlling the vehicle functions is therefore increased further.

The rudiments of the functional relationship, of the temporal relationship and of the local relationship (position and/or direction) explained above can be combined with one another in any desired manner. In addition, the aforementioned examples of the gestures for controlling the vehicle functions can be combined with any desired movements of the wearer of the wearable device. By way of example, a wearer of the wearable device can point to the driver's door, perform a gesture and perform an upward movement. This can be used to initiate closing of the window of the driver's door. The same approach-pointing to the driver's door and performing the same gesture—but with downward movement can be used to initiate opening of the window of the driver's door.

In addition or alternatively, the functional relationship and/or the temporal relationship and/or the local relationship can also be combined with other technologies as desired. By way of example, they may be combined with a voice processing known from the prior art. For this purpose, a microphone can be used in the wearable device. The aforementioned approaches can therefore be combined with voice commands in order to trigger vehicle functions. In the example of the smartwatch, the wearer thereof can form a first near to the vehicle in order to activate a voice processing module in the smartwatch. By way of example, the wearer of the smartwatch can subsequently ask a question, e.g. “What is the state of charge of the vehicle?” and/or “How much longer will the charging process take?”, in response to which the vehicle function may comprise output of the applicable vehicle data. In addition or alternatively, combination with a voice input allows further details to be provided with respect to the vehicle functions by virtue of, in the above example, a gesture for opening the windows, in combination with the voice command “only open the driver's window” being able to trigger opening of the driver's window, whereas the gesture on its own would result in all of the vehicle windows being opened.

According to a second aspect, the underlying object is achieved by a method for the agile, intuitive control of vehicle functions of a vehicle, comprising:
capturing sensor data using a wearable device, the sensor data comprising motion data and optical data relating to a user of the wearable device;
processing the captured sensor data by means of a computing unit;
determining a functional relationship of the processed sensor data with the vehicle by way of the computing unit;
determining an association with a predefinable vehicle function in consideration of the determined functional relationship by way of the computing unit;
controlling or regulating the determined predefinable vehicle function in accordance with the determined association by way of a control unit of the vehicle.

Preferably, the vehicle and the wearable device each comprise a communication unit, the vehicle and the wearable device being designed to set up a Bluetooth Low Energy, BLE, connection to one another.

Preferably, the computing unit is moreover designed to determine a local relationship of the processed sensor data with the vehicle, the determined local relationship being taken into consideration for determining the association with the predefinable vehicle function in consideration of the determined functional relationship.

Preferably, the computing unit is moreover designed to determine a temporal relationship of the processed sensor data with the vehicle, the determined temporal relationship being taken into consideration for determining the association with the predefinable vehicle function in consideration of the determined functional relationship.

Preferably, the temporal relationship comprises the detection of a direction of the detected motion data relating to the user of the wearable device, the detected direction being taken into consideration for determining the association in consideration of the determined functional relationship with the predefinable vehicle function.

These and other objects, features and advantages of the present invention will become clear from studying the detailed description of preferred embodiments that follows and the accompanying figures. It is obvious that—although embodiments are described separately—individual features therefrom can be combined to produce additional embodiments.

### BRIEF DESCRIPTION OF THE DRAWINGS

FIG. 1 schematically shows a system for the agile, intuitive control of vehicle functions of a vehicle; and

FIG. 2 shows an illustrative method for the agile, intuitive control of vehicle functions of a vehicle.

### DETAILED DESCRIPTION OF THE DRAWINGS

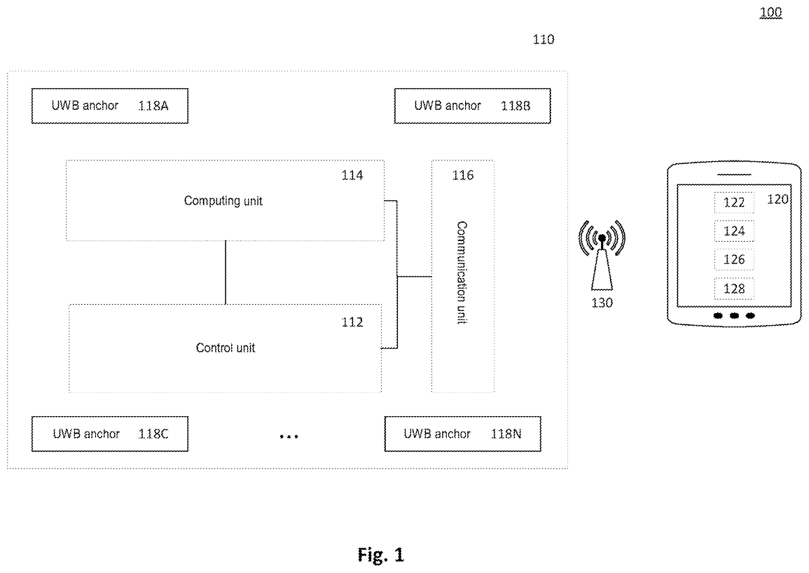

FIG. 1 schematically shows a system 100 for the agile, intuitive control of vehicle functions of a vehicle 110.

The system 100 comprises at least one wearable device 120. Within the context of the document, the term wearable device, or wearable, or wearable computer system 120, covers in particular modern wearable computer systems that are worn on the body of the user of the wearable device 120 during use, in particular smartwatches, but also smartglasses, smartbands, etc., which have a multiplicity of sensors capable of capturing motion data and optical data relating to the wearer of the wearable device 120 and of using a communication unit to transmit them wirelessly—for example using an air or radio interface 130 such as Bluetooth Low Energy (BLE) and/or the mobile radio network.

The wearable device 120 comprises a sensor unit 122 designed to capture sensor data. In particular, the sensor data may comprise motion data relating to the wearer of the wearable device 120. To capture the motion data, the sensor unit 122 can capture sensor data from one or more of the following sensors and may therefore comprise one or more of the following sensors:
an acceleration sensor, or accelerometer, which ascertains an acceleration by measuring an inertial force acting on a mass or test mass, with the result that it can determine the acceleration, a speed increase or decrease and/or a direction of movement of the wearable device 120; and/or
a position determination sensor, or position determination unit, for recording or determining the geographical position, or current position data, using a navigation satellite system. The navigation satellite system may be any established or future global navigation satellite system (GNSS) for position determination and navigation by receiving the signals from navigation satellites and/or pseudolites. By way of example, it may be the Global Positioning System (GPS), GLObal NAvigation Satellite System (GLONASS), Galileo positioning system and/or BeiDou Navigation Satellite System. In the example of GPS, the position determination sensor, or the position determination unit, may comprise a GPS module designed to determine current GPS position data relating to the wearable device 120; and/or
a gyro sensor, which is an acceleration or position sensor designed to record extremely small accelerations, rotational movements and/or a change of position of a mass or test mass. Data from the gyro sensor can be combined with position data from a navigation module, the combination of gyro sensor data and position data being able to be used to ascertain changes of direction, for example, very accurately; and/or
a magnetic field sensor designed to record a current alignment or direction of movement of the wearable device 120; and/or
a proximity sensor for activating and deactivating the display of the wearable device 120; and/or
a UWB sensor system, or UWB sensor, 128 on the wearable device 120 that transmits a signal from the wearable device 120 to multiple UWB anchors, or UWB receivers, 118A . . . 118N in the vehicle 110 and is received on the vehicle 110 at different times depending on the distance from the anchors 118A . . . 118N, the computing unit being able to use this to determine the position of the wearable device 120 relative to the vehicle 110; and/or
at least one further sensor designed to capture motion data from the wearable device.

The sensor data moreover comprise optical data relating to the wearer of the wearable device 120. To capture the optical data relating to the wearer of the wearable device, the sensor unit can capture sensor data from an optical sensor and may therefore comprise an optical sensor. The optical sensor emits light, for example in the green wavelength range, into the tissue, for example on the wrist, and measures the reflected light. The reflected light intensity varies with the pulsation of the blood vessels. This allows the heart rate of the wearer of the wearable device 120 to be determined, for example. In other words, the sensor unit 122 comprises a heart rate sensor that may be designed to measure the heart rate of the wearer of the wearable device.

The system 100 comprises at least one vehicle 110. Within the context of the document, the term vehicle 110 covers mobile means of transport used for transporting people (passenger transport), goods (freight transport) or tools (machines or implements). In particular, the term vehicle 110 covers motor vehicles and also motor vehicles that may be at least partially electrically driven (electric car, hybrid vehicles).

The system comprises at least one computing unit 114, 124. The computing unit 114, 124 is designed to process the captured sensor data. The processing of the sensor data can comprise a preprocessing of the raw sensor data.

In addition, the processing of the sensor data can comprise the determination of a predefinable, or predefined, movement of the user of the wearable device 120, or of a predefinable, or predefined, movement of an arm of the user of the wearable device 120, the wearable device 120 being attached to the arm of the user. Within the context of this document, these data are also called motion data. Examples of a predefined movement as motion data relating to the user of the wearable device 120 may be a step to the side, a jump, a sequence of jumps or any other predefinable, or predefined, movement that can be detected by the sensors.

The predefinable, or predefined, movement of the user of the wearable device 120 can be determined using suitable machine learning algorithms, for example models created using machine learning methods—e.g. by way of supervised learning or unsupervised learning.

In addition or alternatively, the processing of the sensor data can comprise the determination of predefined, or predefinable, gestures of the user or wearer of the wearable device 120. The predefined gestures can be determined using the aforementioned motion data that can be captured by one or more of the aforementioned motion sensors, and also using the optical data from the optical sensor, or optical heart rate sensor, using suitable machine learning algorithms. In particular, the aforementioned sensors can be used to detect subtle differences in a muscle movement and in a tendon activity in the wearer of the wearable device.

Advantageously, this allows the user of the wearable device 120 to use subtle gestures—in the example of the smartwatch—by the hand wearing the smartwatch, for example pressing together the index finger and thumb, closing a fist, etc., to perform a predefined, or predefinable, movement with the least amount of effort.

The predefinable, or predefined, movement, or predefinable, or predefined, gesture thus determined may be associated with a predefinable, or predefined, vehicle function. In addition, some combination of a predefined movement, or predefined gesture, may be associated with a predefined vehicle function. By way of example, the user of the vehicle 110 may stand behind the vehicle 110. The user of the vehicle 110 may wear a smartwatch 120 on one hand. The user of the vehicle 110 may raise this hand to greet a neighbor. This hand movement would not be associated with a predefined vehicle function. If, however, the user of the vehicle 110 performs a predefined hand gesture with this hand and then raises this hand, this combination of gesture and movement may be associated with the vehicle function “open trunk”.

In addition, the computing unit 114, 124 is designed to determine a functional relationship of the processed sensor data with the vehicle 110.

In particular, the functional relationship may arise as a result of one or more of the following conditions:
detected movement, or sequence of movements, of the user of the wearable device 120, as explained hereinabove;
detected gesture, or sequence of gestures, of the user of the wearable device 120, as explained hereinabove.

In addition, the computing unit 114, 124 is designed to determine—when a functional relationship has been detected—an association with a predefined vehicle function. This can be done for example using a continually updatable association stored in a suitable storage unit.

The predefined, or predefinable, vehicle function may comprise one or more of the following vehicle functions:
A. Outside, or exterior of, the vehicle 110:
   open/close a charging flap, or a fuel filler flap, of the vehicle 110;
   unlock a charging connector from a charging socket of the vehicle 110;
   open/close a tailgate of the vehicle 110;
   open/close some flap of the vehicle 110;
   open/close one or more automatic doors of the vehicle 110;
   control a boarding aid of the vehicle 110;
   open/close a garage using a radio interface of the vehicle 110;
   open/close one or more windows of the vehicle 110;
   open/close one or more sunblinds and/or shades of the vehicle 110;
   open/close a sliding and lifting roof of the vehicle 110;
   extend/retract a trailer hitch of the vehicle 110;
   unlock/lock or secure the vehicle 110;
   switch on/off auxiliary heating and/or auxiliary ventilation and/or auxiliary cooling of the vehicle 110;
   switch on/off a parking light and/or sidelight and/or hazard light of the vehicle 110;
activate/deactivate some lighting effect of the vehicle 110;
   trigger a camera recording (photograph or film);
   activate/deactivate a sound or audio output from the vehicle 110;
   actuate a horn of the vehicle 110;
   adjust a seat position and/or steering column of the vehicle 110 with respect to a personalized setting of the wearer of the wearable device 120;
   actuate a level adjustment of the vehicle 110;
   trigger a parking/unparking process of the vehicle 110;
   control a garage parker for automatically parking and unparking the vehicle 110;
   command to stop the vehicle 110 for garage parkers (automatic parking and unparking);
   etc.
B. Inside, or interior of, the vehicle 110:
   scroll, swipe, select and/or slide in the vehicle interior, for example as a substitute for the gesture camera in the vehicle interior;
   take or reject or terminate phone call;
   change the volume of audio outputs in the vehicle 110;
   activate/deactivate a display of a display unit in the vehicle 110;
C. Head-up display of the vehicle 110:
   activate/deactivate a function of the head-up display;
   activate/deactivate a content that is output via the head-up display;
   etc.
D. Status requests with respect to the vehicle 110:
   request a state of charge of an energy store, for example a battery, of an at least partially electrically operated vehicle 110;
   request a tire state of at least one tire of the vehicle 110;
   request a general state of the vehicle 110;
   request a status of the central locking of the vehicle 110;
   request a status of a theft warning system of the vehicle 110;
   request a tank fill level of a tank of the vehicle 110;
   request a range with the current tank fill level and/or the current state of charge of the vehicle 110;
   etc.

The aforementioned predefined, or predefinable, vehicle functions are listed merely by way of illustration; in principle, the approach described in this document allows any desired vehicle functions to be controlled or regulated and/or any desired statuses of the vehicle 110—as listed above—to be called up.

The vehicle 110 comprises a control unit 112. The control unit 112 is designed to control or regulate the predefinable vehicle function in accordance with the determined association.

Advantageously, it is therefore possible to perform particularly agile, flexible and intuitive control and/or regulation of a large number of vehicle functions without the need for expensive sensors and/or operator control elements in the vehicle 110. This facilitates better operator control of functions in and around the vehicle 110 using the latest technologies in the field of wearable devices, which provide the necessary sensors anyway. Another important advantage is that the vehicle functions can be controlled or regulated hands-free, i.e. without input being required from the user of the wearable device 120 using the device 120 itself, since a gesture, for example a hand gesture, of a user of a smartwatch as the wearable device 120 can trigger the aforementioned vehicle function(s) without the need for operator control of the smartwatch using the display. This is particularly convenient for the user of the wearable device 120. New vehicle functions can be added or adapted or modified as desired without the need for complex and expensive adaptations or installations in the vehicle 110.

Preferably, the vehicle 110 and the wearable device 120 each comprise a communication unit 116, 126, the vehicle 110 and the wearable device 120 being designed to set up a Bluetooth Low Energy (BLE) connection to one another.

BLE is a radio technology that allows communication between two communication subscribers. BLE has very low power consumption compared with conventional Bluetooth.

By way of example, the wearable device 120 may already be designed with a function as a digital key, or digital vehicle key, for the vehicle 110 in a manner known from the prior art. BLE technology therefore allows the wearer of the wearable device 1220, without any initial input or request for an initial connection between the vehicle 110 and the wearable device 120, to control or regulate the aforementioned vehicle function(s) hands-free by approaching the vehicle 110 by using a gesture, security with respect to the communication between the vehicle 110 and the wearable device 120 being ensured at the same time by the digital key security requirements.

For the example of a smartwatch as the wearable device 120, it is therefore sufficient for the user of the smartwatch to perform a hand gesture in BLE communication range of the vehicle 110 in order to trigger the aforementioned vehicle function(s). As such, by way of example, the user can form a first with the hand to which the smartwatch is attached in order to unlock and/or lock the vehicle 110. As another example, bringing together the thumb and index finger of the hand wearing the smartwatch can output a state of charge of an energy store of the vehicle 110 on a display of the smartwatch.

Preferably, the computing unit 114, 124 is moreover designed to determine a local relationship of the processed sensor data with the vehicle 110, the determined local relationship being taken into consideration for determining the association with the predefinable vehicle function in consideration of the determined functional relationship.

The data can be transferred from the wearable device 120 to the vehicle 110 using ultra-wideband (UWB) technology, for example. This is a short-range radio communication that uses extremely large frequency ranges with a bandwidth of at least 500 MHz or of at least 20% of the arithmetic mean of the lower and upper cutoff frequencies of the frequency band used. Advantageously, the use of UWB technology allows high-precision determination of the positions of the wearable device 120 in relation to the vehicle 110 to be achieved. The data can be transmitted from the wearable device 120 to the vehicle 110 locally using a suitable radio interface, e.g. Bluetooth Low Energy (BLE). The local relationship can result from the high-precision determination of the position of the wearable device 120, or the wearer of the wearable device 120, in relation to the vehicle 110. The position of the wearable device 120 in relation to the vehicle 110 can be provided in zones in this case, depending on the system design. By way of example, a rear zone, a front zone and side zones can be defined outside the vehicle 110. In addition, the local relationship can also be determined by way of a precise position of the wearable device 120 with respect to the vehicle 110, for example 1 meter (m) in front of the driver's door of the vehicle 110. One alternative is for it to also be possible for the local relationship in the vehicle interior to be determined as a precise position in the vehicle interior. In another example, the vehicle interior can also be divided into zones, e.g. driver's seat, front-seat passenger's seat, backseat area on the right, etc.

The inclusion of the local relationship increases the agility and flexibility of the control of the vehicle functions further because, in the example of the smartwatch as the wearable device 120, the wearer thereof can use a predefinable hand gesture, for example, to initiate opening of the driver's door while standing on the driver's side of the vehicle 110, to initiate opening of the front-seat passenger's door while standing on the front-seat passenger's side of the vehicle 110, to initiate opening of the trunk while standing at the rear of the vehicle 110, etc.

Preferably, the computing unit 114, 124 is moreover designed to determine a temporal relationship of the processed sensor data with the vehicle 110, the determined temporal relationship being taken into consideration for determining the association with the predefinable vehicle function in consideration of the determined functional relationship.

By way of example, the temporal relationship may comprise the determination of a duration of a gesture of the wearer of the wearable device 120 and/or of a temporal variable, for example a start time and a stop time. In the example of the smartwatch, the wearer thereof can use a hand gesture, for example, to initiate opening of a vehicle window, relaxation of the hand gesture resulting in the process being terminated, which means that opening of the vehicle window is terminated when the hand gesture is relaxed, and the window is opened only to produce a state desired by the user of the smartwatch. This increases the agility and flexibility of the control of the vehicle functions further.

In addition or alternatively, the temporal relationship can also be used to attain functional safety goals. A functional safety goal may require the correct functionality of a vehicle function to be assured along the whole process chain thereof. In a conventional vehicle electrical system communication, this may be an Alive signal. The Alive signal is a signal from the wearable device 120 that cyclically changes its value in order to signal to the vehicle 110 that the wearable device 120 is still “alive”. This allows the vehicle 110 to ensure that a signal status, for example relating to a hand gesture from the wearable device 120, is not “frozen” in the process chain.

For the example of the smartwatch, the wearer thereof can initiate the start of an action, for example an automated parking process for the vehicle 110, by forming a fist, for example. The functional safety goal may now require, during the automated parking process for the vehicle 110, as the functional safety goal, the wearer of the smartwatch to cyclically bring together their thumb and index finger and relax them again during the automated parking process in order to signal that the automated parking process is intended to be continued. If the aforementioned functional safety goal (cyclically bringing together and relaxing the thumb and index finger) is not met, the automated parking process would be terminated for safety reasons. By forming a first again, the wearer of the smartwatch can trigger the end of the action, here the automated parking process. Such an approach demonstrates agile combinational control of vehicle functions.

Advantageously, the agility and flexibility of the control of the vehicle functions is therefore increased further, safety when controlling the vehicle functions being ensured at the same time.

Preferably, the local relationship moreover or alternatively comprises the detection of a direction of the motion data relating to the user of the wearable device 120, the detected direction being taken into consideration for determining the association with the predefinable vehicle function in consideration of the determined functional relationship.

For this purpose, the aforementioned UWB technology can be used. UWB technology allows high-precision determination of not only the positions of the wearable device 120 in relation to the vehicle 110 but also a direction of a movement of the wearable device 120 in relation to the vehicle 110. By way of example, the direction of the motion data relating to the user of the wearable device 120 can therefore be determined, or detected, by using UWB technology in a manner known from the prior art. In the example of the smartwatch as the wearable device 120, the wearer of the smartwatch can for example use a hand gesture and point to the rear door on the driver's side of the vehicle 110 in order to initiate opening of the window on the rear driver's side door of the vehicle 110. By using the same hand gesture and pointing to the front driver's side door of the vehicle 110, the wearer can initiate opening of the window of the front door on the driver's side of the vehicle 110.

Advantageously, the agility and flexibility for controlling the vehicle functions is therefore increased further.

The rudiments of the functional relationship, the temporal relationship and the local relationship (position and/or direction) explained above can be combined with one another in any desired manner. In addition, the aforementioned examples of the gestures for controlling the vehicle functions can be combined with any desired movements of the wearer of the wearable device 120. By way of example, a wearer of the wearable device can point to the driver's door, perform a gesture and perform an upward movement. This can be used to initiate closing of the window of the driver's door, for example. The same approach-pointing to the driver's door and performing the same gesture—but with downward movement can be used to initiate opening of the window of the driver's door.

In addition or alternatively, the functional relationship and/or the temporal relationship and/or the local relationship can also be combined with other technologies as desired. By way of example, they may be combined with a voice processing known from the prior art. For this purpose, a microphone can be used in the wearable device 120. The aforementioned approaches can therefore be combined with voice commands in order to trigger vehicle functions. In the example of the smartwatch, the wearer thereof can form a first near to the vehicle 110 in order to activate a voice processing module in the smartwatch. By way of example, the wearer of the smartwatch can subsequently ask a question, e.g. “What is the state of charge of the vehicle?” and/or “How much longer will the charging process take?”, in response to which the vehicle function may comprise output of the applicable vehicle data. In addition or alternatively, combination with a voice input allows further details to be provided with respect to the vehicle functions by virtue of, in the above example, a gesture for opening the windows, in combination with the voice command “only open the driver's window” being able to trigger opening of the driver's window, whereas the gesture on its own would result in all of the vehicle windows being opened.

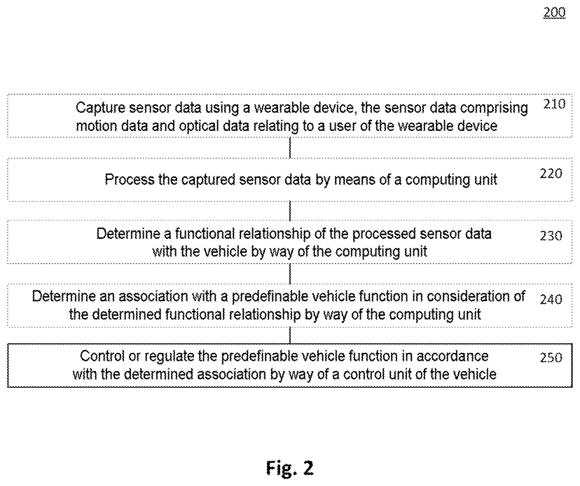

FIG. 2 shows a method 200 for the agile, intuitive control of vehicle functions of a vehicle that can be carried out by a system 100 as described with reference to FIG. 1.

The method 200 comprises:
capturing 210 sensor data using a wearable device 120, the sensor data comprising motion data and optical data relating to a user of the wearable device 120;
processing 220 the captured sensor data by means of a computing unit 114, 124;
determining 230 a functional relationship of the processed sensor data with the vehicle 110 by way of the computing unit 114, 124;
determining 240 an association with a predefinable vehicle function in consideration of the determined functional relationship by way of the computing unit 114, 124;
controlling or regulating 250 the determined predefinable vehicle function in accordance with the determined association by way of a control unit 112 of the vehicle 110.

The vehicle 110 and the wearable device 120 may each comprise a communication unit 116, 126, the vehicle 110 and the wearable device 120 being designed to set up a Bluetooth Low Energy, BLE, connection to one another.

The computing unit 112, 122 may moreover be designed to determine a local relationship of the processed sensor data with the vehicle 110, the determined local relationship being taken into consideration for determining the association of the processed sensor data with the predefinable vehicle function.

The computing unit 112, 122 may moreover be designed to determine a temporal relationship of the processed sensor data with the vehicle 110, the determined temporal relationship being taken into consideration for determining the association of the processed sensor data with the predefinable vehicle function.

The local relationship may comprise the detection of a direction of the captured motion data relating to the user of the wearable device 120, the detected direction being taken into consideration for determining the association of the processed sensor data with the predefinable vehicle function.

1.-10. (canceled)

11. A system for agile, intuitive control of vehicle functions of a vehicle, comprising:
a wearable device designed to capture sensor data, the sensor data comprising motion data and optical data relating to a user of the wearable device;
a computing unit configured to:
process the captured sensor data;
determine a functional relationship of the processed sensor data with the vehicle; and
determine an association with a predefinable vehicle function in consideration of the determined functional relationship,
wherein the vehicle comprises a control unit designed to control or regulate the predefinable vehicle function in accordance with the determined association.

12. The system according to claim 11, wherein
the vehicle and the wearable device each comprise a communication unit, and
the vehicle and the wearable device are configured to set up a Bluetooth Low Energy connection to one another.

13. The system according to claim 11, wherein
the computing unit is further configured to:
determine a local relationship of the processed sensor data with the vehicle, the determined local relationship being taken into consideration for determining the association with the predefinable vehicle function in consideration of the determined functional relationship.

14. The system according to claim 11, wherein
the computing unit is further configured to:
determine a temporal relationship of the processed sensor data with the vehicle, the determined temporal relationship being taken into consideration for determining the association with the predefinable vehicle function in consideration of the determined functional relationship.

15. The system according to claim 13, wherein
the local relationship comprises the detection of a direction of the motion data relating to the user of the wearable device, the detected direction being taken into consideration for determining the association with the predefinable vehicle function in consideration of the determined functional relationship.

16. The system according to claim 13, wherein
the computing unit is further configured to:
determine a temporal relationship of the processed sensor data with the vehicle, the determined temporal relationship being taken into consideration for determining the association with the predefinable vehicle function in consideration of the determined functional relationship.

17. A method for agile, intuitive control of vehicle functions of a vehicle, comprising:
capturing sensor data using a wearable device, the sensor data comprising motion data and optical data relating to a user of the wearable device;
processing the captured sensor data via a computing unit;
determining a functional relationship of the processed sensor data with the vehicle via the computing unit;
determining an association with a predefinable vehicle function in consideration of the determined functional relationship via the computing unit;
controlling or regulating the determined predefinable vehicle function in accordance with the determined association by way of a control unit of the vehicle.

18. The method according to claim 17, wherein
the vehicle and the wearable device each comprise a communication unit, and
the vehicle and the wearable device are designed to set up a Bluetooth Low Energy connection to one another.

19. The method according to claim 17, further comprising:
determining a local relationship of the processed sensor data with the vehicle, the determined local relationship being taken into consideration for determining the association with the predefinable vehicle function in consideration of the determined functional relationship.

20. The method according to claim 17, further comprising:
determining a temporal relationship of the processed sensor data with the vehicle, the determined temporal relationship being taken into consideration for determining the association with the predefinable vehicle function in consideration of the determined functional relationship.

21. The method according to claim 20, wherein
the temporal relationship comprises detecting a direction of the detected motion data relating to the user of the wearable device, the detected direction being taken into consideration for determining the association in consideration of the determined functional relationship with the predefinable vehicle function.

22. The method according to claim 19, further comprising:
determining a temporal relationship of the processed sensor data with the vehicle, the determined temporal relationship being taken into consideration for determining the association with the predefinable vehicle function in consideration of the determined functional relationship.
